(19) United States
(12) Patent Application Publication
(10) Pub. No.: US 2021/0019977 A1
(43) Pub. Date: Jan. 21, 2021

(54) VENDING MACHINE FOR DIALYSIS PATIENTS

(71) Applicant: Jorge Serna, El Paso, TX (US)

(72) Inventor: Jorge Serna, El Paso, TX (US)

(21) Appl. No.: 16/511,266

(22) Filed: Jul. 15, 2019

(51) Int. Cl. G07F 9/00; G05B 15/02; G16H 20/60; G07F 11/00

(52) U.S. Cl. CPC G07F 9/006; G07F 11/00; G16H 20/60; G05B 15/02

(57) ABSTRACT

A vending machine for dispensing a customized dietary supplement to dialysis patients, is disclosed. The vending machine comprises a serving dispenser designed to hold and distribute the customized dietary supplement to a user. The vending machine further comprises a user interface, a controller having a memory unit to store an inventory of ingredients operatively linked to a user interface and programmed to control the serving dispenser. The controller is configured to dispense the customized dietary supplement based on a user input. The vending machine prevents user from having unhealthy snacks for dialysis. The method decreases health complication faced by the dialysis patients. The vending machine provides wide product selection and optimal component storage capacities. It also affords improved product quality, which helps in improving the patients' health.

### BACKGROUND OF THE INVENTION

A. Technical Field

The invention disclosed herein generally relates to a vending machine for dialysis patients. More particularly it relates to a vending machine which dispense customized dietary supplements based on user's choice.

B. Description of Related Art

Generally, vending machines helps in selling pre-packaged foods and beverages, which are known in the art. It is generally known that in a vending system a user can view available food items through a display window and select the desired food item. Selection and dispensing process occur in response to direct consumer input and without any need for a manual assistance. By eliminating the human intermediary, vending machines have the benefit of permitting unattended sales at all hours. Vending machines provide a unique and required market for selling food or snack items, whether solid food or drink, in a local environment, offices, hospital, etc. Vending machines are also known, which dispense beverages or solid foods where the customer selects the combinations thereby the machine dispenses selected components from storage containers in measured amounts to the customer.

There are about 12,000 dialysis clinics around the country. Patients with end stage kidney disease require hemodialysis treatment three times a week for about 4 hours. Users are frequently getting snacks from vending machines in the dialysis units with items that are harmful for these patients. Vending machines with snacks targeted for people with advance kidney disease on dialysis are less in the market. Food or liquid beverages obtained from the vending machines available in the dialysis center are not recommended for their health.

Henceforth, there is a need for vending machines with snacks, drinks, protein supplements, vitamin supplement and nutritional supplements that are rich in nutrition recommended for this particular group of individuals.

### SUMMARY OF THE INVENTION

The present invention relates to a vending machine dispensing protein supplements, vitamin supplement and nutritional supplements for a dialysis patient. A method for selection of customized dietary supplement with a vending machine is provided. The method also includes receiving a user input of a customized dietary supplement into a user interface of the vending machine. Additionally, the method includes a controller and a memory unit for storing the inventory list and its ingredients. The method also includes the controller receiving the user input and fetching the inventory list from the memory unit and determining the customized dietary supplement based on the user's input. The method also includes dispensing the customized dietary supplement to the user.

In one embodiment, a system for selecting of customized dietary supplement is provided. The system includes a serving dispenser that dispenses customized dietary supplement. The system comprises a user interface of the vending machine for receiving a user input of a customized dietary supplement. Additionally, the system includes a controller and a memory unit for storing the inventory list and its ingredients. The controller of the vending machine receives the user input and fetches the inventory list from the memory unit and determine the customized dietary supplement based on the user input. The serving dispenser dispense the customized dietary supplement to the user.

In one embodiment, the customized dietary supplement depends at least in part on one or more of the following items: low potassium, low phosphorus, low sodium, low calories, low fat, saturated fat, low cholesterol, carbohydrates, low sugar, and high protein. In a preferred embodiment, the customized dietary supplement depends on the number of calories of the products.

In one embodiment, the user input is the technology associated with the vending machine by which the user inputs an indication of his product selection. The user interface may be, for example, buttons, knobs, a key pad, or a touch screen display.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF DRAWINGS

The foregoing summary, as well as the following detailed description of the invention, is better understood when read in conjunction with the appended drawings. For the purpose of illustrating the invention, exemplary constructions of the invention are shown in the drawings. However, the invention is not limited to the specific methods and structures disclosed herein. The description of a method step or a structure referenced by a numeral in a drawing is applicable to the description of that method step or structure shown by that same numeral in any subsequent drawing herein.

FIG. 1 illustrates a schematic view of a vending machine in one embodiment of the present invention.

FIG. 2 is a flow chart illustrating exemplary method of using vending machine in one embodiment of the present invention.

### DETAILED DESCRIPTION OF EMBODIMENTS

A description of embodiments of the present invention will now be given with reference to the Figures. It is expected that the present invention may be embodied in other specific forms without departing from its spirit or essential characteristics. The described embodiments are to be considered in all respects only as illustrative and not restrictive.

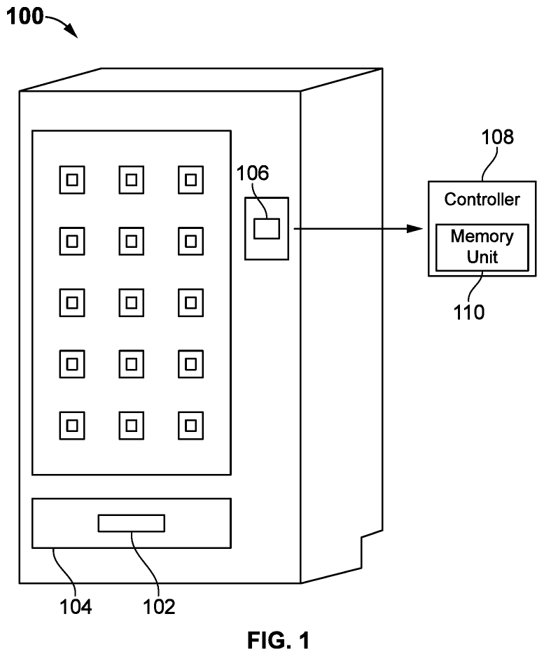

Referring to FIG. 1, the present invention discloses a vending machine 100 dispensing protein supplements, vitamin supplement and nutritional supplements for dialysis patient. The vending machine 100 includes one or more food and/or beverage products which could be a customized dietary supplement 102. The vending machine 100 also includes a user interface 106 adapted to allow a user or customer to select the customized dietary supplement 102. The user interface 106 is operably connected to a controller 108. A user inputs his selection into the vending machine 100 using the user interface 106 such as series of buttons, a key pad, touch screen, or other input mechanism. A serving dispenser 104 dispenses the customized dietary supplement 102 which could be a food and/or beverage product. The vending machine 100 also includes a controller 108 operatively linked to the user interface 106. The controller 108 is programmed to control the serving dispenser 104. The controller 108 having a memory unit 110 to store an inventory of ingredients. The controller 108 controls the serving dispenser 104 to dispense the customized dietary supplement 102 based on the user input. The user could then retrieve the customized dietary supplement 102 from the serving dispenser 104. The serving dispenser 104 is at least partly operated by the user or by an operator.

In one embodiment, the memory unit 110 stores inventory of ingredients compositions and their properties. The memory unit 110 also store ingredients including the nutrition data. The controller 108 have its own memory unit 110 to store the programs, but could either use its own memory to store data or could use external memory of the vending machine 100. For example, the memory unit 110 may be stored at a remote or virtual facility (e.g. cloud computing). The memory unit 110 stores a nutrition parameter to generate a customized dietary supplement 102.

In one embodiment, a method for selection of customized dietary supplement 102 with a vending machine 100 is provided. The method also includes receiving a user input of a customized dietary supplement 102 into a user interface 106 of the vending machine 100. Additionally, the method includes a controller 108 and a memory unit 110 for storing the inventory list and its ingredients. The method also includes the controller 108 receiving the user input and fetching the inventory list from the memory unit 110 and determining the customized dietary supplement 102 based on the user input. The method also includes dispensing the customized dietary supplement 102 to the user.

In one embodiment, the customized dietary supplement 102 depend at least in part on one or more of the following items: low potassium, low phosphorus, low sodium, low calories, low fat, saturated fat, low cholesterol, carbohydrates, low sugar, and high protein. In a preferred embodiment, the customized dietary supplement 102 depends on the number of calories of the products. In a preferred embodiment the customized dietary supplement 102 is protein powder. In a preferred embodiment, the customized dietary supplement 102 depends on the number of calories of the products.

In one embodiment, the customized dietary supplement 102 is selected from a group comprising protein supplements, vitamin supplements, nutritional supplement, mineral supplements, herbal supplements, antioxidants, edible fiber additives, fruit products; edible seeds; nuts, nut products, and mixtures thereof.

In one embodiment, the customized dietary supplement 102 is selected from a group consisting of: high fiber, low fat, high protein, soy protein, low carbohydrate, organic, low potassium, low phosphorus, low sodium, low sugar, low calorie, vitamin enhanced, and creatine supplement.

In one embodiment, the user interface 106 is a graphical interface responsive to receive a user input. The user input is the technology associated with the vending machine 100 by which the user inputs an indication of his product selection. The user interface 106 may be, for example, buttons, knobs, a key pad, or a touch screen display. the user interface 106 displays a price for customized dietary supplement 102.

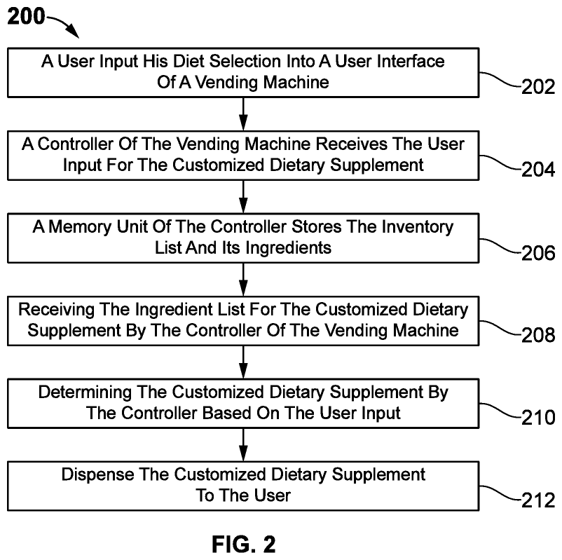

Referring to FIG. 2 is a flow chart illustrating exemplary method of the vending machine 100 in one embodiment of the present invention. A vending machine 100 is configured to perform a process 200 which includes the following steps (i) A user input his diet selection into a user interface of a vending machine, at step 202, (ii A Controller of the vending machine receives the user input for the customized dietary supplement, at step 204, (iii) A memory unit of the controller stores the inventory list and its ingredients, at step 206, (iv) Receiving the ingredient list for the customized dietary supplement by the controller of the vending machine, at step 208, (v) Determining the customized dietary supplement by the controller based on the user input at step 210, and (vi) Dispense the customized dietary supplement to the user, at step 212. The flow diagrams described herein (e.g., of the method 200) do not necessarily imply a fixed order to the actions (unless otherwise specified), and embodiments may be performed in any order that is practicable.

In one embodiment, the process or method 200 described herein may be performed by hardware, software (including microcode), firmware, or any combination thereof. For example, a memory unit 110 may store thereon instructions that when executed by a machine result in performance according to any of the embodiments described herein.

In one embodiment, a system for selecting of customized dietary supplement 102 in the vending machine 100 is provided. The customized dietary supplement 102 aims at the nutritional and metabolic needs of dialysis patients. The system includes a serving dispenser 104 that dispenses customized dietary supplement 102. The system comprises a user interface 106 of the vending machine 100 for receiving a user input of a customized dietary supplement 102. Additionally, the system includes a controller 108 and a memory unit 110 for storing the inventory list and its ingredients. The controller 108 of the vending machine 100 receives the user input and fetches the inventory list from the memory unit 110 and determine the customized dietary supplement 102 based on the user input. The serving dispenser 104 dispense the customized dietary supplement 102 to the user.

In one embodiment, the vending machines 100 incorporating the system could be located primarily in hospitals especially in dialysis center but not limited to retail chain stores, in shopping malls, supermarkets, convenience stores, universities, schools, gymnasiums, workplaces, stadiums, theatres, or any other place where potential customers are likely to be seeking for the customized dietary supplement 102.

In one embodiment, the customized dietary supplement 102 could be of low calories, high protein, no trans-fat; low saturated fat, dietary fiber etc. The vending machine 100 could dispense snacks, drinks, protein supplements, vitamin supplement, and nutritional supplements that are rich in ingredients recommended for dialysis patients. The vending machine 100 customized dietary supplement 102 are designed to fit kidney patient diet which is of high protein, low potassium, low phosphorus and low sodium.

In one embodiment, the customized dietary supplement 102 could also be used as nutritional supplements for patients undergoing dialysis therapy or for patients on a restricted diet. In addition, the customized dietary supplement 102 can be used to treat the nutritional deficiencies of any disease state that results in increased oxidative stress, elevated cholesterol levels, or elevated homocysteine levels.

The vending machine 100 prevents user from having unhealthy snacks for dialysis. The method 200 decreases health complication faced by the dialysis patients. The vending machine 100 provides wide product selection and optimal component storage capacities. It also affords improved product quality, which helps in improving the patients' health.

Preferred embodiments of this invention are described herein, including the best mode known to the inventors for carrying out the invention. It should be understood that the illustrated embodiments are exemplary only and should not be taken as limiting the scope of the invention.

The foregoing description comprise illustrative embodiments of the present invention. Having thus described exemplary embodiments of the present invention, it should be noted by those skilled in the art that the within disclosures are exemplary only, and that various other alternatives, adaptations, and modifications may be made within the scope of the present invention. Merely listing or numbering the steps of a method in a certain order does not constitute any limitation on the order of the steps of that method. Many modifications and other embodiments of the invention will come to mind to one skilled in the art to which this invention pertains having the benefit of the teachings presented in the foregoing descriptions. Although specific terms may be employed herein, they are used only in generic and descriptive sense and not for purposes of limitation. Accordingly, the present invention is not limited to the specific embodiments illustrated herein.

What is claimed is:

1. A vending machine for dispensing a customized dietary supplement, comprising:
a serving dispenser designed to hold and distribute the customized dietary supplement, said vending machine comprises a user interface; and
a controller having a memory unit to store an inventory of ingredient operatively linked to a user interface and programmed to control the serving dispenser;
wherein the controller is configured to dispense the customized dietary supplement based on a user input.

2. The vending machine of claim 1, wherein the customized dietary supplement is selected from a group comprising protein supplements, vitamin supplements, nutritional supplement, mineral supplements, herbal supplements, antioxidants, edible fiber additives, fruit products; edible seeds; nuts, nut products, and mixtures thereof.

3. The vending machine of claim 1, wherein the memory unit stores a nutrition parameter to generate a customized dietary supplement.

4. The vending machine of claim 1, wherein the serving dispenser is at least partly operated by the user or by an operator.

5. The vending machine of claim 1, wherein the customized dietary supplement is selected from a group consisting of: high fiber, low fat, high protein, soy protein, low carbohydrate, organic, low potassium, low phosphorus, low sodium, low sugar, low calorie, vitamin enhanced, and creatine supplement.

6. The vending machine of claim 1, wherein the customized dietary supplement is protein powder.

7. The vending machine of claim 1, wherein the memory unit stores inventory of ingredients compositions and their properties.

8. The vending machine of claim 1, wherein the user interface is a graphical interface responsive to receive a user input.

9. The vending machine of claim 1, wherein the user interface displays a price for customized dietary supplement.

10. The vending machine of claim 1, wherein the user interface is selected from a group of a button, a knob, a key pad, or a touch screen display.
